(19) United States
(12) Patent Application Publication
(10) Pub. No.: US 2008/0038784 A1
(43) Pub. Date: Feb. 14, 2008

(54) Method Of Converting A Biomass Into Biobased Products

(76) Inventor: Christopher D'Arnaud-Taylor, New York, NY (US)

Correspondence Address: GREENBERG TRAURIG, LLP, MET LIFE BUILDING, 200 PARK AVENUE, NEW YORK, NY, 10166, US

(21) Appl. No.: 11/573,250

(22) PCT Filed: Aug. 4, 2005
(86) PCT No.: PCT/US05/27721

§ 371 Date: Aug. 2, 2007

Related U.S. Application Data
(60) Provisional application No. 60/598,880, filed on Aug. 4, 2004.

(51) Int. Cl. C12P 7/10; C12P 19/04; C12P 7/08

(52) U.S. Cl. 435/101; 435/163; 435/165

(57) ABSTRACT

The present invention provides a method of converting a biomass into biobased products. An impact process of accelerating and decelerating the biomass is engaged to fractionate the biomass. As the biomass is fractionated, it is also classified by each resulting biomass fraction. The biomass fraction is then processed into a biobased product.

### CROSS REFERENCE TO RELATED APPLICATIONS

This application is based upon provisional application Ser. No. 60/598,880 entitled METHOD OF CONVERTING A BIOMASS INTO BIOBASED PRODUCTS, the entirety of which is incorporated herein by reference.

### BACKGROUND OF THE INVENTION

The world today is facing growing burdens caused by overpopulation, depletion of fossil fuels, increasing demands for fuels, pollution of air, water and land, global warming and climate changes, forest cover destruction, and agricultural land loss. Although to some extent some of these concerns can be met through the improved use of solar energy and windpower and increased nuclear power, more conservation of resources and more efficient use of resources are always being sought.

Fibrous cellulosic material, such as straw, corn stalks (stover), bagasse, hardwoods, cotton stalks, kenaf and hemp, are composed primarily of cellulose (typically, 40-60% dry weight), hemicellulose (typically 20-40% by dry weight) and lignin (typically 5-25% by dry weight). These components, if economically separated fully from one another, can provide vital derivative sources of fermentable sugars for the production of alcohols, ethers, esters, and other chemicals. There is a growing interest in the manufacture of biobased products such as biofuels from cellulosic biomass by fermentation with enzymes or yeast. As used herein, biofuels refers to fuel such as ethanol for the generation of electricity and for transportation. Biofuels are beneficial in that they add fewer emissions to the atmosphere than petroleum fuels. They also are beneficial in that they use herbaceous and sparsely used woody plants and, particularly, plant wastes that currently have little or no use. Biofuels are obtained from renewable resources and can be produced from domestic, readily available plants and wastes, thus reducing dependence on coal, gas and fossil fuel in addition to boosting local and world-wide economies.

There is a need for an economical method for cleanly separating the basic components of fibrous, ligno-cellulosic materials and the fermentable sugars they represent from one another. In particular, it has proved difficult to economically separate the mixed hexose and pentose structured hemicellulose from the lignin and other, minor, components, such as lipids and silica, present in biomass. The processes which exist today focus on techniques such as ball-milling, two-roll milling, cryogenic grinding, explosive depressurization, ultrasonics and osmotic cell rupture followed by ethanol extraction, as well as conventional pulping techniques. All use high levels of technology, fossil energy and investment and, accordingly, are expensive and, often, highly polluting. For example, conventional pulping processes, which use high temperatures (e.g., 175.degree. C.) and pressure (e.g., 175 psi) and sulfite, kraft or alkali to obtain purified cellulose, known as alpha pulp, are well recognized as involving high investment, energy and operating costs, including recovery of chemicals.

In spite of these difficulties, interest in finding alternative methods for fractionating cellulosic biomass remains high, since fibrous cellulosic vegetation constitutes a vast renewable potential source of energy from plant sugars as ethanol and of animal food. Worldwide, it is estimated that there are available over twenty billion tons annually of agricultural fibrous waste that could provide a source of cellulose, hemicellulose and lignin. In the U.S., wheat straw and corn stover constitute some billion tons annually, much of which is wasted.

### SUMMARY OF THE INVENTION

The present invention provides a method of converting a biomass into biobased products. In particular it is directed a method for fractionating and separating a biomass safely to separate the cellulosic, hemicellulosic and lignin components of fibrous plant materials. The separated components are useful for the production of fossil fuel derivatives, biodegradable plastics, edible protein, and a variety of other products. A biomass is conditioned such that it can be fractionated into any number of fractions. An impact process is engaged to fractionate the biomass. The biomass is fractionated such that at least one of the biomass fractions comprises a base element of said biomass. Once the biomass is fractionated, it is separated by each resulting biomass fraction. The biomass fraction is then processed into a biobased product.

A biomass may comprise base elements such as cellulose, hemicellulose, lignin, and ash. Cellulose can be processed to produce the biomass product ethanol. Xylitol can be produced from processed hemicellulose. Lignin may be processed to produce any number of value added products.

### BRIEF DESCRIPTION OF THE DRAWINGS

FIG. 1 is a block flow diagram showing the overall method wherein a biomass is converted to biobased products.

FIG. 2 is a block flow diagram showing the method for conditioning the biomass for fractionation.

FIG. 3 is a block flow diagram showing the method for fractionating the biomass.

FIGS. 4, 5, and 6 are block flow diagrams showing the method for processing each of the fractionated biomasses (hemicellulose, cellulose, and lignin respectively).

### DETAILED DESCRIPTION OF THE INVENTION

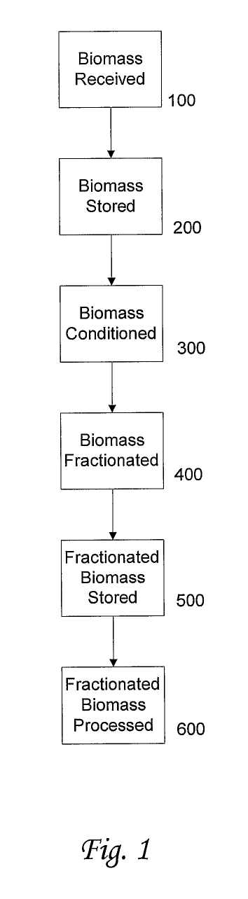

FIG. 1 is a flow chart showing the overall method wherein a biomass is converted to biobased products, including such exemplary products as ethanol, xylitol, and other value added products. It is pointed out that the method of the invention is described using non-limiting examples, none of which limit the scope of the invention herein disclosed. A biomass refers to any plant derived organic matter, including crops and trees dedicated to energy purposes or otherwise, agricultural food and feed crops, agricultural crop wastes and residues, wood wastes and residues, aquatic plants, animal wastes, municipal wastes, and other waste materials.

Initially a biomass is received 100 for storage and processing. The biomass can be brought and received by a number of transport methods and in a number of forms. A number of non-limiting examples are provided. A vehicle designed to release payload from its bottom such as a “hopper truck” may be used to dump a loose biomass from its bottom. A “walking floor” truck may use a moving floor to discharge the loose biomass while the trailer of the truck remains level. A “tip truck” may also be used. This truck uses hydraulics to lift its trailer bed and dump the loose biomass from either the tipped trailer, or just the trailer bed.

A biomass can be received in any number of forms. For example, rather than being loose, a biomass may also be bailed or wrapped. Such biomass can be packaged in varied forms (e.g.: round, square, or stacked). When this is the case, the biomass is lifted by a separate lifting device or vehicle, such as a forklift, from the delivery vehicle.

The biomass is then stored while it awaits processing, as shown at step 200. Again, multiple non-limiting forms of storage may be used for the various forms in which biomass is received. A biomass that is received in a loose or free flowing form requires a substantial degree of protection from uncontrolled environmental conditions. A steel or concrete silo may be used as, for example, one with environmental controls for dust, odor, and humidity. The silo can be designed with protective devices and functionality to protect against dangers such as fire and combustion. A concrete bunker may also be utilized with loose biomass. The bunker can be used with a loose biomass that is to be covered and contained but requires a front end loader to transport. Such a bunker is designed with concrete walls and floors that protect from wicking moisture from the ground. A flexible, waterproof cover protects against environmental conditions such as wind-erosion and precipitation. A concrete floor and cover may be used for storing packaged biomass such as that which is baled or wrapped. The baled biomass can be stacked one atop the other and covered to protect against, for example, precipitation.

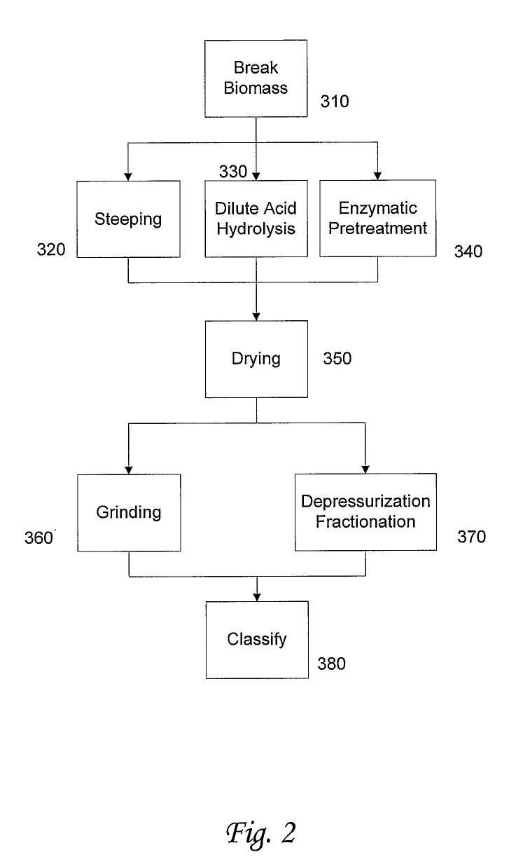

The biomass remains in storage until such time as it to be conditioned for fractionation, fermentation, or both. Biomass conditioning 300 is shown in greater detail at FIG. 2. Starting at 310, the biomass is broken down to reduced sizes for feeding into the conditioning systems described below. The gross biomass feedstock, be it either bailed or whole unit loose biomass (e.g.: corn cobs, corn stalks, switch grass, or straw) is shredded or otherwise broken.

After breaking the biomass, a series of optional conditioning pretreatments may also be performed. For example, biomass feed stocks may be soaked in water for a period of time. An exemplary, non-limiting time may be for anywhere from hours to a number of days. This steeping step 320 can be useful in fractionation or fermentation, these steps of the process being discussed further below. The biomass may also be treated with a dilute mineral or organic acid. This dilute acid hydrolysis 330 aids in opening up certain base structures of the biomass; this in turn furthers downstream fractionation or fermentation. Finally, an enzymatic pretreatment 340 may be used to condition the biomass. This process is similar to the dilute acid hydrolysis, but it alters different biomass base structures, allowing them to be further processed downstream.

Before the broken down or shredded biomass can be introduced to the grinding 360 step, and ultimately a fractionation system and process, the biomass must be dried to a target of 15% moisture content. After drying to the target moisture, the biomass is ground 360. Grinding renders the biomass into a particulate form. The particle dimension can be about 3 millimeters or less. A grinder such as a hammer mill or an attrition mill can be used.

As an alternative to grinding, a depressurization fractionation 370 may be employed to further particulate the biomass where one of the optional conditioning pretreatment steps 320,330,340 has been preformed. Biomass moisture content achieved during the drying 350 for this alternative can be set independently for optimal processing.

At 380 is shown that a classification step can be implemented to classify the particle size from the ground, or the depressurization characteristic, of the biomass. For example, larger biomass particles may be fed back into the grinder if necessary. Any device or method may be used to check or separate the larger particles. It can be classified using a shaker with a screen that allows certain sized particles through, or may simply undergo a quality assurance check.

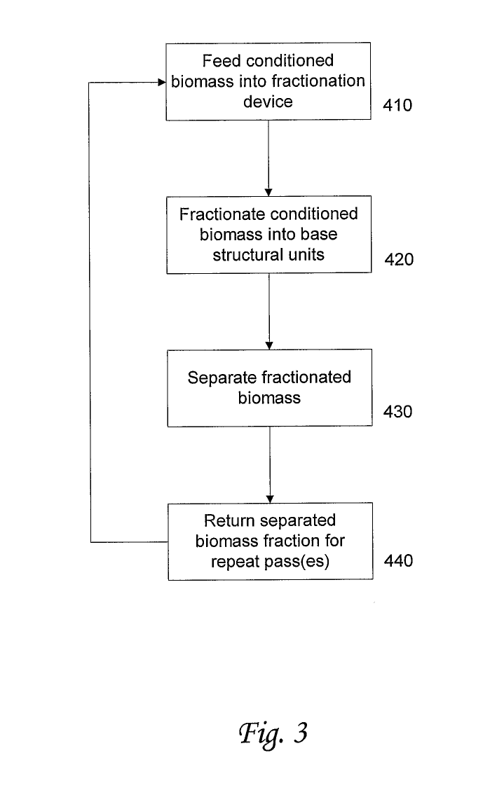

Turning back to FIG. 1, the method moves from biomass conditioning 300 to fractionation of the conditioned biomass 400. An impact, or force-based fractionation process can be used. At FIG. 3, the fractionation of the conditioned biomass is shown. The conditioned biomass may be fractionated by being fed into a fractionation system 410. A fractionation system is regulated so that the conditioned biomass is inputted at a desired mass flow rate. Various devices such as alarms and interlocks may be used to monitor the flow rate to assure consistency.

In order to break down the conditioned biomass into the base structural units 420 comprising it (for example cellulose/fiber, hemicellulose, lignin, and ash), low shear, high impact zones are used throughout the fractionation system. The fractionation process is employed with only minimal temperature increases. This preserves the chemical composition of the conditioned biomass. Any devices used to carry out the fractionation are comprised of impact structures that allow the biomass to be collided such that it is crushed through the application of motive force that causes the biomass to disaggregate into its base structures. Non-limiting exemplary methods and apparatuses, which may be used to achieve fractionation and separation in the present invention, are described in U.S. Patent Publication No. 2003/0221997, the entirety of which is incorporated herein.

One exemplary, non-limiting method of fractionation making use of low shear, high impact zones include those using separators. Among the various types of separators, pneumatic separators, i.e., ones with forced fluid flow for the drawing-along (entrainment) of the material, may be used. In the context of the above-mentioned pneumatic separators, they are separators of particulate material made up of a plurality of cyclone devices set in series, in which the mixture of materials is introduced into a container having the shape of a truncated cone with a vertical axis (cyclone), usually in a direction tangential to the side walls of the latter, so as to obtain a centrifugal vertical flow of the material to be separated. The biomass particles, which are induced, in their circular motion, to slide along the side walls of the container, are thus substantially subject to the centrifugal force resulting from the flow of conveying air, to the force of friction, in a direction opposite to the centrifugal force, which develops in the interaction of the material with the walls of the container themselves, and to the force of gravity. Inside the cyclone there is also present an ascending flow of air, which develops at the vertical axis of the cyclone itself.

The different kinetic energy which, by virtue of the above-mentioned forces, particles with different density and particle-size possess brings about a separation of the material within the cyclone, whereby the particles of large weight tend to drop along the walls and to deposit in a collection hopper, which is set at the base of the container, the said container having the shape of a truncated cone, whereas the finer particles, which are of small weight, tend to be drawn by the forced flow of air towards an outlet pipe, which is usually axial, of the cyclone itself. The geometry of the container having the shape of a truncated cone and the amount of flow of drawing air determine separation of particles that are of different particle-sizes (i.e. granulometry). Hence, by using in series cyclones presenting different characteristics and possibly varying the characteristics of the flow, a progressive classification of the particles is obtained.

The cyclone devices may also comprise internal portions which act as collision surfaces upon which the conditioned biomass impacts, thus breaking biomass into the base structures comprising it. For example, a sliding support member that is adapted to direct a flow of fluid (e.g.: air) that further aids in classifying particles may also provide an impact surface against which the conditioned biomass collides and breaks into its constituent parts. The conditioned biomass is fractionated such that a particle's density is a function of its base structure units. For example, cellulose is at a different particle density than hemicellulose, and both are at yet another different particle density than lignin.

Conditioned biomass may be fractionated by other devices, and may be included in or work in conjunction with devices such as the exemplary separator. For example, a grinding device could be employed to achieve one degree of fractionation, and the cyclonic devices comprising the impact surfaces could provide the other degrees of fractionation.

A series of separation zones can be connected in series to separate the fractionated biomass components 430. Non-limiting exemplary methods and apparatuses, which may be used to achieve separation in the present invention, are again described in U.S. Patent Publication No. 2003/0221997. For example, in above-mentioned device useful for carrying out the process, a series of cyclones, also described above, may be employed to cast out heavier particles first, with each cyclone depositing progressively finer particulate material. The final cyclone, exemplifying the last zone, deposits the lightest and least dense fractionated biomass particles. The separation is carried out by virtue of a flow of fluid—air for instance—which is adapted to facilitate fine separation of the conditioned biomass. The fractionated biomass may be returned from the separated streams back through the fractionation process for further processing of desired 440.

Returning once again to the overall method as depicted in FIG. 1, the fractionated biomass is stored for further processing, as shown in block 500. Non-limiting examples of storage for fractionated biomass take the forms of either wet or dry storage. Dry storage may take the form of standard silo or elevator design with dust and explosion control, as well as a “live bottom” for discharging stored material.

Wet storage may be implemented where it is desirable to remove impurities from the biomass fractions. Washing steps for removing impurities or water-soluble components from biomass fractions can be used in wet storage. Wet storage may be employed when enzymatic pretreatment steps, as described in FIG. 2, 340, are desired to reduce the downstream processing steps and fermentation times.

Two non-limiting examples of wet storage are a slurry tank or a wet bunker. In a wet bunker, the biomass fractions are stored in uncovered basins. Water can be pumped through the top of the fraction pile. The water drains through the biomass fraction pile, whereupon the drainage is recovered at the base of the pile. Impurities or water-soluble components can be removed from the drainage water, after which the water may be pumped into the biomass, repeating the process. An additional biomass fraction of the same type can be slurried and pumped onto the top of the pile for ease of transport as well as further washing.

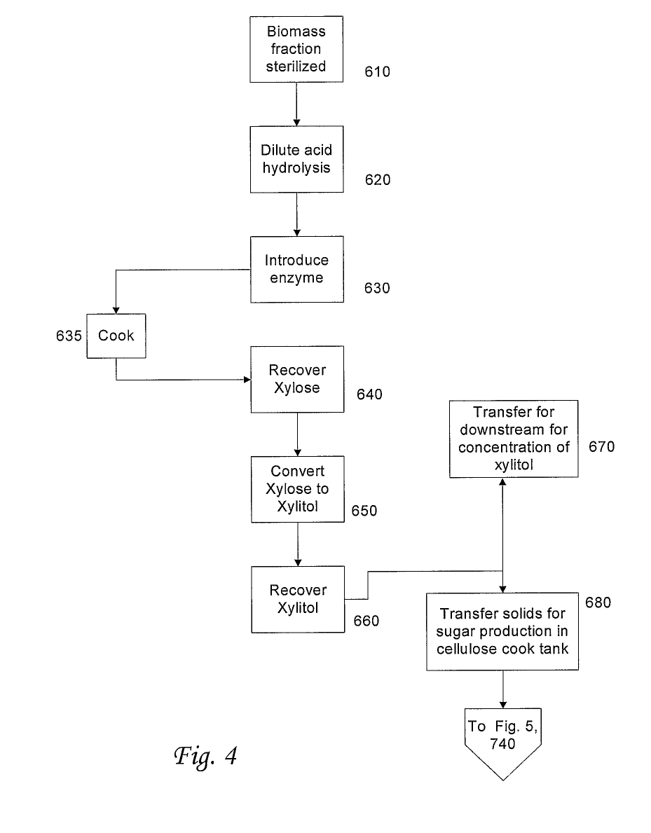

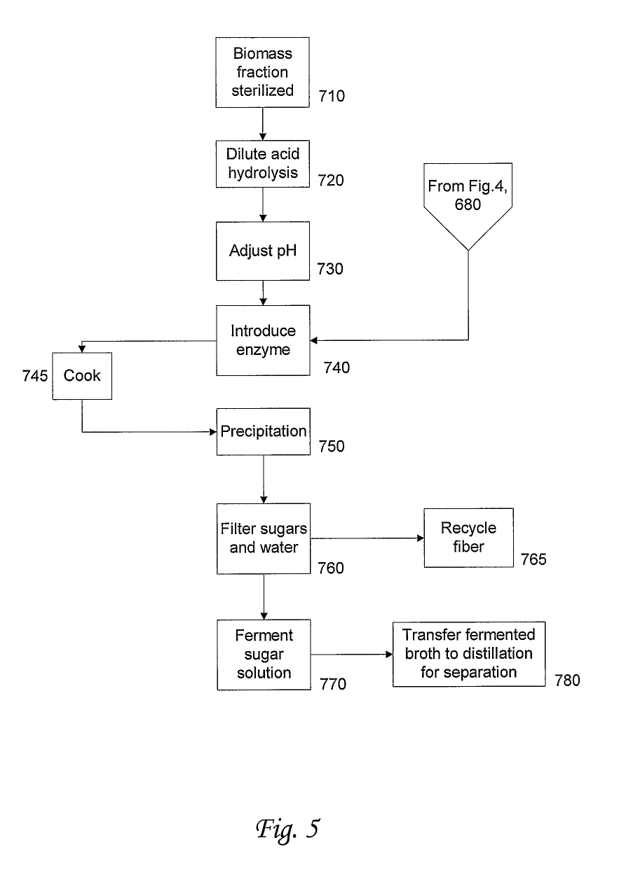

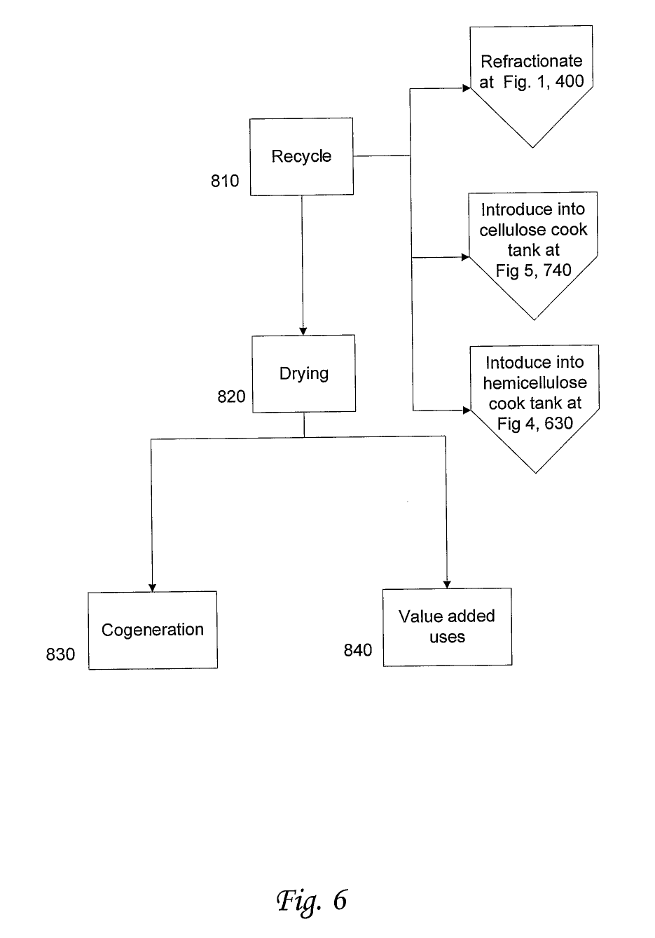

The overall method shown at FIG. 1 moves to processing each of the various biomass fractions 600. FIGS. 4 to 6 describe the methods for processing the biomass fractions. FIG. 4 shows the method of processing a hemicellulose-rich biomass fraction, FIG. 5 the cellulose-rich biomass fraction process, and FIG. 6 the lignin-rich fraction process.

The hemicellulose-rich biomass fraction (hereinafter, “hemicellulose fraction”) is processed as shown in FIG. 4. First, the hemicellulose fraction is sterilized 610. Sterilization may be employed in order to eliminate undesired microbial or fungal growth that can impede further processing or result in lower yields of the final product. Sterilization methods may include temperature-based method such as pasteurization. Chemicals may also be added to carry out the sterilization process.

Dilute acid hydrolysis 620 can be employed in the next step. The dilute acid hydrolysis can be implemented to convert some of the pentose trapped in the hemicellulose fraction into fermentable sugar. The hydrolysis may also assist in the removal of inorganic matter.

An enzyme adapted to produce xylose from the hemicellulose fraction when it is cooked is added 630. A xylanase enzyme may be introduced into a tank containing the hemicellulose fraction. Introduction of the enzyme should be at pH, temperature and solids loading parameters appropriate to the enzyme supplied. The hemicellulose fraction can then be cooked 635 such that xylose is produced in a cooked broth. Once the xylose is produced, it may be recovered 640 utilizing an appropriate separation technique to separate xylose from the cooked broth.

The recovered xylose sugar solution can then be fermented such that it produces xylitol 650. The xylose sugar solution is combined with nutrients and yeast/microbes that are adapted to convert xylose to xylitol. By following the appropriate fermentation temperature and pH profile, xylitol is produced. Once the xylitol is produced, an appropriate separation technique may be utilized to recover a xylitol solution 660, which may then be transferred to a downstream process for concentrating xylitol 670. At 680, the remaining solids not transferred for xylitol concentration may be used in the method for processing the cellulose-rich biomass fraction shown in FIG. 5 at step 740, wherein the solids are added to a cellulose cook tank for the production of sugars for ethanol. Otherwise the solids may be disposed of.

As shown in FIG. 5, the method for processing the cellulose-rich biomass fraction, (hereinafter, the “cellulose fraction”), begins with a sterilization process 710 similar to the one for processing the hemicellulose fraction shown in FIG. 4, 610.

Dilute acid hydrolysis 720 follows, where it is also employed to convert trapped pentose to fermentable sugar and to assist in removing inorganic matter. At step 730 the pH is adjusted, using either lime or sodium, prior to the introduction of an enzyme or microbe. The pH is adjusted using the guidelines appropriate to the supplied enzyme or microbe.

Once the pH is adjusted, the enzyme is added 740 to a cook tank holding the cellulose fraction. The solids recovered from the processing of the hemicellulose fraction FIG. 4, 680 may also be added to the tank for the production of sugars for ethanol. The enzymes (here being cellulose) adapted to produce the sugars are introduced at the proper temperature, pH and solids loading. The cellulose fraction thus treated is then cooked 745 at parameters adapted to produce fermentable sugars.

A precipitation step 750 follows the cooking. An acid, base, or water polymer treatment may be added to resultant cellulose cook to assist in settling solids. This may be employed to facilitate the recycling of cellulose as well as undigested fiber. The settling of solids may also result in higher fermentation rates, as inert matter and material-inhibiting fermentations can be removed prior to fermentation.

A filtration step 760 may be used to assure that a sugar solution consisting essentially of water and sugars passes to the fermentation stage of the process. This may also be employed to facilitate recycling of the enzymes, cellulose, and the undigested fiber 765. As stated earlier, the removal of the inert matter and material which inhibits fermentation results in higher fermentation rates.

At 770 fermentation of the sugar solution is shown. The sugar solution from the processed cellulose fraction is combined with yeast/microbes adapted to produce ethanol. Fermentation is carried out following the appropriate fermentation temperature and pH profile parameters. Once fermentation is complete, the fermentation broth can be transferred to a distillation stage 780 for separating out ethanol.

The lignin-rich biomass fraction is processed as shown at FIG. 6. A recycling step may be employed, as shown at 810. Depending upon the composition and value of the lignin stream, varied recycling options may be implemented. For example, the lignin fraction may be refractioned FIG. 1, 400, introduced into the hemicellulose cook tank for cooking FIG. 4, 630 or similarly introduced into the cellulose cook tank FIG. 5, 740. Any lignin which is recovered from cooks or fermentation broths can be concentrated and dried 820 to a desired specification. For example, at a cogeneration step 830, any lignin to be used for energy production is to be dried to less than 50% moisture content.

The processed lignin fraction may also be further processed for value added uses 840, such as binders, adhesive additives, fertilizer additives, seed coats, or any number of uses desired by an end-user. Where the lignin is put to such uses, an end user may set the processing guidelines for pH, moisture and particle size.

Although the present invention has been described in relation to particular preferred embodiments and examples thereof, many variations and modifications and other uses may be made without departing from the invention. Accordingly, it is intended that all such alterations and modifications be included within the spirit and scope of the invention as disclosed herein. Further, all examples provided herein do not limit the scope of the invention.

1. A method of converting a biomass into a biobased product comprising:
conditioning a biomass such that it can be fractionated;
impacting said biomass into biomass fractions such that each biomass fraction comprises a base structure of the biomass;
separating the biomass fractions by the base structure; and
processing a separated biomass fraction such that a biobased product is produced.

2. The method of claim 1 wherein the biomass comprises the base structures of cellulose, hemicellulose, and lignin.

3. The method of claim 2 wherein a biomass fraction comprising the base structure of cellulose is processed to produce at least one biomass product comprising ethanol.

4. The method of claim 3 wherein the processing of the fraction comprising base structure of cellulose comprises:
sterilizing the cellulose fraction;
employing dilute acid hydrolysis;
adjusting the pH appropriate to an enzyme adapted to produce fermentable sugars from the cellulose fraction when cooked;
adding the enzyme;
cooking the cellulose fraction to a cellulose cook such that it produces fermentable sugars;
precipitating the cellulose cook;
filtering inert matter from the cellulose cook;
fermenting a sugar solution resulting from the filtered cellulose cook; and
distilling ethanol from the fermented sugar solution.

5. The method of claim 4 wherein the processing further comprises:
adding a solid material recovered from a processed hemicellulose biomass fraction prior to cooking the fraction to produce fermentable sugars.

6. The method of claim 2 wherein the biomass fraction comprising the base structure of hemicellulose is processed to produce at least one biomass product comprising xylitol.

7. The method of claim 6 wherein the processing of the fraction comprising base structure of hemicellulose comprises:
sterilizing the hemicellulose fraction;
employing dilute acid hydrolysis;
adjusting the pH appropriate to an enzyme adapted to produce xylose from the hemicellulose fraction when it is cooked;
adding the enzyme; and
cooking the hemicellulose fraction to produce xylose in a cooked broth;
separating a xylose sugar solution from the cooked broth;
fermenting the xylose sugar solution such that it converts xylose to xylitol; and
separating a xylitol solution.

8. The method of claim 7 wherein method further comprises:
transferring the xylitol solution to a downstream process for concentrating xylitol.

9. The method of claim 7 wherein method further comprises:
recovering any remaining solid material such that it can be used for processing a cellulose biomass fraction.

10. The method of claim 2 wherein the biomass fraction comprising the base structure of lignin is processed to produce at least one biomass product comprising at least one value added product.

11. The method of claim 10 wherein the processing of the fraction comprising the base structure of lignin comprises recycling a lignin stream, including:
a) refractionating the lignin stream;
b) introducing the refractionated lignin into:
i) a hemicellulose cook tank; or
ii) a cellulose cook tank;
c) recovering the lignin from i) or ii); and
d) concentrating and drying recovered lignin to meet a use specification.

12. The method of claim 11 wherein the use specification for the recovered lignin includes use for energy production, the recovered lignin being dried to less than about 50% moisture content.

13. The method of claim 10 wherein in value added product is selected from the group consisting of:
binders;
adhesive additives;
fertilizer additives; and
seed coats.

14. A method of converting a biomass into a biobased product comprising:
a first step of fractionating said biomass into a plurality of fractions such that each biomass fraction comprises a base structure of said biomass;
a second step of separating each biomass fraction; and
a third step of processing each biomass fraction such that a biobased product is produced.

15. The method of claim 14 wherein the biomass comprises the base structures of cellulose, hemicellulose, and lignin.

16. The method of claim 15 wherein the biomass fraction comprising the base structure of cellulose is processed to produce at least one biobased product comprising ethanol.

17. The method of claim 16 wherein the biomass fraction comprising the base structure of lignin is processed to produce at least one biomass product comprising at least one value added product.

18. The method of claim 17 wherein in the value added product is selected from the group consisting of:
binders;
adhesive additives;
fertilizer additives; or
seed coats.

19. The method of claim 15 wherein the biomass fraction comprising the base structure of hemicellulose is processed to produce at least one biomass product comprising xylitol.
